(19) United States
(12) Patent Application Publication
(10) Pub. No.: US 2009/0148493 A1
(43) Pub. Date: Jun. 11, 2009

(54) METHODS AND DEVICES FOR ENHANCED BIOCOMPATIBILITY

(75) Inventors: Ralph Ballerstadt, Portland, OR (US); George Jackson, Pearland, TX (US)

Correspondence Address: BIO TEX, INC., 8058 EL RIO STREET, HOUSTON, TX, 77054, US

(73) Assignee: BIOTEX, INC., Houston, TX (US)

(21) Appl. No.: 12/329,527

(22) Filed: Dec. 5, 2008

Related U.S. Application Data
(60) Provisional application No. 60/992,646, filed on Dec. 5, 2007.

(51) Int. Cl. A61F 2/00; B05D 1/00; A61K 31/7088; A61K 31/7105; A61K 31/711

(52) U.S. Cl. 424/423; 427/2.24; 514/44

(57) ABSTRACT

The present invention is directed to devices with enhanced biocompatibility and methods for generating and utilizing such devices. The present invention is further directed to enhanced biocompatibility utilizing oligonucleotide functionalization. In one aspect, a device for implantation and/or prolonged exposure to the body tissues includes a functionalized surface. The functionalized surface generally enhances the biocompatibility of the device with body tissues. In some embodiments, the functionalized surface includes substances for controlling interaction between the device and the body tissues. Substances for controlling interactions may include, but are not limited to, polymeric materials, biomolecules, ions and/or ion-releasing substances, and/or any other appropriate substance or combination thereof. In exemplary embodiments, the functionalized surface includes oligonucleotides for controlling interaction between the device and the body tissues. In some exemplary embodiments, the oligonucleotides are aptamers.

### CROSS-REFERENCE TO RELATED APPLICATIONS

This application claims the benefit of U.S. provisional patent application Ser. No. 60/992,646, filed Dec. 5, 2007, entitled “METHODS AND DEVICES FOR ENHANCED BIOCOMPATIBILITY”, the entire contents of which are hereby incorporated by reference.

### FIELD OF THE INVENTION

The present invention is related to implantable devices with enhanced biocompatibility and methods for generating and utilizing such devices. The present invention is further related to enhanced biocompatibility utilizing oligonucleotide functionalization.

### BACKGROUND OF THE INVENTION

Operational functionality of long-term implants over more than 30 days depends on the biocompatibility properties of the outer implant materials. Inadequate materials tend to elicit a foreign body reaction leading to fibrotic capsule formation which insulates the device from the surrounding tissue. To enable implants to communicate with tissue over long period of time, such hostile reactions need to be prevented. Current focus in medicine and biology is to engineer materials that pro-actively control the interaction of the material surface with the biological milieu comprised of a medley of cells, proteins, and ions. Such control could ultimately lead to implants with long-term operational functionality due to the absence of a foreign body reaction. Various polymers (PEG, dextran) have been identified and successfully studied as potential antifouling coatings. However, providing a comprehensive battery of cell-specific ligands remains a challenge.

One approach is directed at coating sensors with materials that mimic the NO-releasing properties of endothelial cells, by which platelet adhesion and activation may be inhibited and vasoconstriction around the implant may be minimized.

Another approach is focused on a class of cell-adhesion proteins found in extracellular matrix containing the three amino acid sequence ArgGlyAsp (RGD) which bind to particular cell surface receptors (e.g. integrins). Much research has been conducted to examine the effects of adsorbing RGD-containing proteins and immobilizing short synthetic RGD-containing peptides to model substrates to help mediate adhesion, spreading, and phenotypic expression. These efforts have been partially successful. However, this approach has various pitfalls. Often only one particular class of receptors is pursued, ignoring potential other type of protein-cell and cell-cell specific interactions. Peptides are further very expensive to isolate or synthesize, raising the cost for research tremendously. Peptides are also prone to enzymatic attack, minimizing the overall biochemical stability of this approach. Peptides can also be immunogenic which may require immunogenically matching the source or the properties of the synthesized peptide with the one of the host. Overall, the approach to employ peptides for enhancing biocompatibility of sensor implants has significant shortcomings (expense, limited biochemical diversity, chemical instability, immuno-type matching).

### SUMMARY OF THE INVENTION

The present invention is directed to implantable devices with enhanced biocompatibility and methods for generating and utilizing such devices. The present invention is further directed to enhanced biocompatibility utilizing oligonucleotide functionalization.

In one aspect, a device for implantation and/or prolonged exposure to the body tissues includes at least a portion of at least one functionalized external surface. The functionalized surface or portions thereof generally enhances the biocompatibility of the device with body tissues. In some embodiments, the functionalized surface includes substances for controlling interaction between the device and the body tissues. Such substances for controlling interactions may include, but are not limited to, polymeric materials, biomolecules, ions and/or ion-releasing substances, and/or any other appropriate substance or combination thereof. In some exemplary embodiments, the functionalized surface includes at least one unique molecule, for controlling interaction between the device and the body tissues. Unique molecules may include oligonucleotides, nucleic acid constructs, and/or any other similar or appropriate molecules. Oligonucleotides are short, typically single-stranded nucleic acids. In some exemplary embodiments, the oligonucleotides are, for example, aptamers or the oligonucleotides may include aptamers as at least a portion of the sequences of the oligonucleotides.

Aptamers are short RNA, DNA-based nucleotide sequences or combination thereof that are developed in vitro by combinatorial chemistry library approaches, generating potential cell-specific ligands in large scale which may be screened rapidly for affinity to particular cell types, representative of specific body tissues. The limited biochemical diversity of nucleotides compared with amino acids in proteins is offset by the large complexities of libraries of potential cell-specific aptamers that can be easily produced and investigated. Lack of knowledge of the identity and/or abundance of the effective target may not be a disadvantage, as the binding of an aptamer to unknown cell receptors may also increase the potential number of sensor/cell interactions for improved implant acceptance. Another advantage of aptamers is their intrinsically low immunogenicity and good chemical stability against nuclease attack. The systematic evolution of aptamer ligands may be facilitated by exponential enrichment utilizing systematic evolution of ligands by exponential enrichment (SELEX) protocols. SELEX methods are based on repeated rounds of in vitro selection of oligonucleotide ligands followed by their amplification. Oligonucleotide sequences with appropriate binding affinity to a target may then be utilized as aptamers.

In exemplary embodiments, the surface of the device may be functionalized by providing aptamers selected for binding to a large number of structural components of the extracellular matrix (ECM). In one exemplary embodiment, for example, the diversity of the aptamers may approach the diversity of the binding sites found in the ECM. In other embodiments, at least one aptamer for an ECM component may be utilized.

The ECM has numerous functions, including providing structural support, segregating neighboring tissues, and mediating intercellular communication. It is generally composed of an interlocking network of fibrous proteins and glycosaminoglycans including a large number of proteoglycans.

In some embodiments, a diverse library of aptamers may be included that that selectively binds to the diverse binding sites of body tissues and/or components thereof. Attachment of a device to the body tissues, particularly to the ECM and/or cell surface may thus be utilized to promote biocompatibility of the device, decrease the immune response from the host, promotes cell adhesion and cell growth, and accelerates tissue restoration. This may therefore result in mitigating responses such as, for example, fibrotic capsule formation as often observed with current approaches. The device may then remain and continue to operate in the body tissues for extended periods of time.

Aptamers may generally be produced via a Selective Evolution of Ligands by Exponential Enrichment (SELEX) protocol and may be selected against biological matter, such as the body tissue, the ECM or components thereof. In general, body tissue or samples thereof of an intended patient may be utilized during the selection process to aid in optimum binding affinity. While aptamers are analogous to antibodies in their range of target recognition and variety of applications, they also possess several key advantages over their protein counterparts. For example, they are smaller, easier and more economical to produce, are capable of greater specificity and affinity, are highly biocompatible and non-immunogenic, and can easily be modified chemically to yield improved properties. After selection, aptamers may also be produced by chemical synthesis, which may eliminate batch-to-batch variation that may complicate production of therapeutic proteins.

In some exemplary embodiments, SELEX may be performed to generate aptamers utilizing a whole-cell or whole-tissue approach. This may be desirable, as whole-cell or whole-tissue targets may present appropriate target molecules in a “native” state. Further, multiple targets may be present in such samples and may thus be utilized to increase the diversity of the generated aptamer pool. In some embodiments, non-whole-cell or non-whole-tissue targets may also be utilized which may include, but are not limited to, purified molecular samples, anchored target molecules, artificial micelles and/or liposomes presenting target molecules, and/or any other appropriate target.

In one aspect, attaching a diverse library of aptamers to at least a portion of one surface of a device may be accomplished. The diversity of the library may also approach the diversity of complementary binding targets in a tissue component.

In another aspect, a device may be functionalized with, for example, aptamers so that it may be present at tissue boundaries during implantation. The aptamers may be chosen in complementary to the differences in the tissue composition across the boundary. Spatial placement of substances such as aptamers, may be arranged on the surface of a device to aid in attachment of the device to the body tissue. For example, when a device is to be present at a boundary between two layers or regions of tissue with different ECM compositions, one portion or side of the device may be functionalized with one set of substances (e.g. aptamers) selected for binding to one layer or region of tissue and the other portion or side of the device may be functionalized with a different set of aptamers or other molecules for binding to the other layer or region.

In some embodiments, aptamers for different ECM components may be included in proportions relative to abundance of its target in the body tissue. For example, aptamers selected to bind to a more common ECM component, such as collagen, may be included in a higher proportion on the device than aptamers selected against a rarer ECM component, or vice versa. The proportionality of the aptamers may be chosen for an appropriate application. For example, the device may be functionalized to bind to different layers or regions of tissue utilizing the same ECM molecular composition that may only vary in relative abundance of ECM components across a boundary by, such as, varying the relative abundance of aptamers on the surface of the device. This may, for example, include utilizing a higher abundance of an aptamer on one portion of the device and a lower abundance on another portion such that the higher abundance portion may bind to a corresponding tissue region or layer with a higher abundance of a target ECM component.

In general, substances may be included on the device surface by an appropriate method, such as, for example, adsorption, precipitation, ionic attachment, covalent attachment, and/or any other appropriate attachment method.

The present invention together with the above and other advantages may best be understood from the following detailed description of the embodiments of the invention illustrated in the drawings.

### BRIEF DESCRIPTION OF THE FIGURES

FIG. 1 illustrates the general concept of generating specific aptamers by SELEX;

FIG. 1a illustrates generating aptamers from a sample including multiple binding targets;

FIG. 1b illustrates the interaction of a device including aptamers with a biological material;

FIG. 2 illustrates a method of improving the biocompatibility of an implantable device;

FIGS. 3a, 3b, 3c, 3d and 3e illustrate multimeric and chimeric aptamers;

FIG. 4 the relative binding affinities of sets of aptamers to fibronectin;

FIG. 5 shows the results of a spot binding assay of a set of aptamers to fibronectin, BSA and milk proteins; and

FIG. 6 shows the attachment of aptamers to a cellulose membrane.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention is directed to devices, for example, implantable devices, with enhanced biocompatibility to biological tissue and methods for generating and utilizing such devices. The present invention is further directed to enhanced biocompatibility utilizing, for example, oligonucleotide functionalization, on at least at least a portion of the surface of the device.

In one aspect, a device for implantation and/or prolonged exposure to the body tissues may include at least a portion of a functionalized surface. The functionalized portion of the surface generally enhances the biocompatibility of the device with body tissues. In some embodiments, the functionalized surface includes substances for controlling interaction between the device and the body tissues, particularly camouflaging the unmodified or chemical surface of the device. The substances for controlling interactions may include, but are not limited to, polymeric materials, biomolecules, ions and/or ion-releasing substances, and/or any other appropriate substance or combination thereof. In some exemplary embodiments, the at least one portion of functionalized surface includes oligonucleotides for controlling interaction between the device and the body tissues. In some exemplary embodiments, the oligonucleotides may be aptamers.

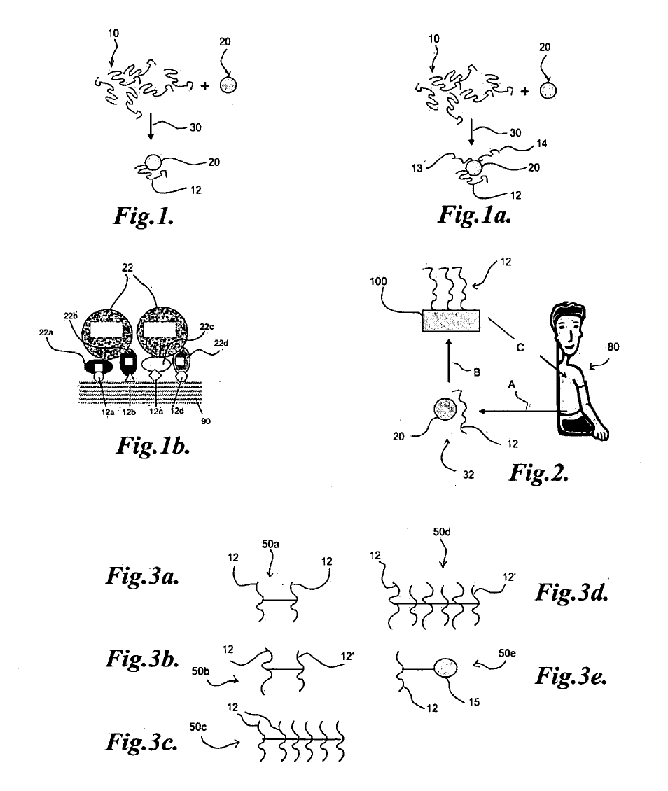

As shown as in FIG. 1, aptamers may generally be produced via a Selective Evolution of Ligands by Exponential Enrichment (SELEX) protocol and may be selected against the ECM or components thereof. In general, it may be desirable to utilize at least one of the body tissue or sample thereof of an intended patient during the selection process to aid in optimum binding affinity. As illustrated, the SELEX technique may begin with a large library 10 of random single stranded nucleotide aptamers which may be a DNA, RNA or combination thereof. The library 10 is then exposed 30 to a target 20 and the aptamers 12 bound to the target 20 are separated and amplified for the next round. The binding conditions for each round may be made more stringent than in the previous round until the only remaining aptamers in the pool are highly specific for and bind with high affinity to the target 20. While aptamers are analogous to antibodies in their range of target recognition and variety of applications, they possess several key advantages over their protein counterparts. For example, also, as noted above, they are smaller, easier and more economical to produce, are capable of greater specificity and affinity, are highly biocompatible and non-immunogenic, and can easily be modified chemically to yield improved properties. After selection, aptamers may also be produced by chemical synthesis, which may eliminate batch-to-batch variation that may complicate production of therapeutic proteins.

In some exemplary embodiments, SELEX may be performed to generate aptamers utilizing a whole-cell or whole-tissue approach. This may be desirable as whole-cell or whole-tissue targets may present appropriate target molecules in a “native” state. Further, multiple targets may be present in such samples and may thus be utilized to increase the diversity of the generated aptamer pool or library 10. In some embodiments, non-whole-cell or whole-tissue targets 20 may also be utilized which may include, but are not limited to, purified molecular samples, anchored target molecules, artificial micelles and/or liposomes presenting target molecules, and/or any other appropriate target. As illustrated in FIG. 1a, targets 20 may include multiple components for binding aptamers 12, 13, 14, for example. The aptamers 12, 13, 14, for example, may each bind separate targets on the diverse target 20.

In some embodiments, aptamers may be included that selectively bind to body tissues and/or components thereof. Attachment of a device to the body tissues, particularly to the extracellular matrix (ECM) and/or cell surface may thus be utilized to promote biocompatibility of the device by promoting cell adhesion and cell growth, and decrease the immune response from the host, and by, for example, accelerating ECM tissue restoration. The device may then remain and continue to operate in the body tissues for extended periods, for example, several weeks to months. As illustrated in FIG. 1b, the surface 90 may be functionalized with a plurality of aptamers, such as aptamers 12a, 12b, 12c and 12d. The aptamers may then bind to targets in the body tissue, such as, for example, ECM molecules 22a, 22b, 22c and 22d on cells 22, as illustrated.

In exemplary embodiments, the surface of the device may be functionalized by providing aptamers selected for binding to a large number of structural components of the ECM. In one exemplary embodiment, the diversity of the aptamers may approach and/or exceed the diversity of the binding sites found in the ECM. In other embodiments, at least one aptamer for an ECM component may be utilized. The diversity of binding sites may, for example, be between 1 and 1,000,000. In some embodiments, a desirable diversity of aptamers utilized may be between 1 and 1,000.

The ECM has numerous functions, including providing structural support, segregating neighboring tissues, and mediating intercellular communication. It is generally composed of an interlocking network of fibrous proteins and glycosaminoglycans including a large number of proteoglycans. Many cells bind to components of the extracellular matrix. This process is regulated by integrins, specific cell surface proteins that dock cells to ECM structures or to different integrin proteins on the surface of other cells. For example, the attachment of fibronectin to the extracellular domain triggers intracellular signaling pathways as well as the association with the cytoskeleton via a set of adaptor molecules such as actin. By providing as many interfacial cues on the surface of device as possible to the highly complex extracellular matrix, the device may better integrate into the body tissues and remain for extended periods for operation.

In an exemplary embodiment, at least a portion of the surface of the device may be functionalized by coating with aptamers selected for binding to the ECM. The aptamers may generally be produced via a SELEX protocol and may be selected against the ECM or components thereof. In general, it may be desirable to utilize the body tissue of an intended patient during the selection process to aid in selectivity and specificity. Components of the ECM used for selection may include, but are not limited to, fibrous proteins such as fibronectin, laminin, elastin, vitronectin and collagen, proteglycans such as heparan sulfate and chondroitin sulfate, and/or any other appropriate component of the ECM or combination thereof. It may generally be desirable to include aptamers selected against a variety of components of the ECM and in particular components of the ECM related to the type of body tissue with which the device may interact. Other targets, such as cell surface receptors, whole cell/tissue samples and/or any other appropriate target or combination thereof may be utilized to generate aptamers. Aptamers selected with sufficiently high affinity for a target may be produced by an appropriate method, such as chemical synthesis and/or biosynthesis, and may then be coated onto or attached to the device.

FIG. 2 illustrates a method for functionalizing and implanting a device 100 in a patient 80. As shown, a target, for example, tissue sample, 20 may be taken A from a patient 80. Aptamers 12 may then be generated against the sample 20, such as by a SELEX procedure 32. The aptamers 12 may then be attached B to the device 100, which may then be implanted C into the patient 80.

In some embodiments, DNA aptamers may be utilized as they are generally more stable than RNA and are generally less susceptible to degradation. DNA 3′-exonuclease activity may be prevalent in body tissues, and thus DNA aptamers may be further screened for stability in body tissues by, for example, incubation in serum and/or other enzymatic treatment. To further stabilize immobilized aptamers from enzymatic attack in the tissue, the surface may be complemented with polymeric molecules that may sterically block enzymes from degrading the aptamers. Such polymeric molecules may be biocompatible synthetic polymers, such as PEG (polyethylene glycol), polyurethane, or silanes, natural polymers, such as dextrans, or other polysaccharides, and/or any other appropriate material. Further, the aptamers may also be modified, such as 2′-RNA modifications, N3′-P5′ phosphoramidates, modified sugar moieties (such as 2′deoxy-2′fluoro-b-D-arabino nucleic acids), 3′-dideoxy bases, 3′-inverted bases, and/or any other appropriate modifications or combinations thereof.

In some embodiments, aptamers for different ECM components may be included in proportions relative to the abundance of their targets in the body tissue. For example, aptamers selected to bind to a more common ECM component, such as collagen, may be included in a higher proportion on the device than aptamers selected against a less abundant ECM component, or vice versa. In general, the proportionality of the aptamers may be chosen for an appropriate application.

In some embodiments, aptamers and/or other substances may be included to aid in reducing biofilm formation and/or otherwise reduce the presence of infectious agents. Aptamers may, for example, be selected for low/no affinity to microbial organisms. Other aptamers may be utilized that bind to microbial organisms and colocalize a biocidal substance, such as, for example, a quaternary ammonium moiety, to the microbial organisms.

In general, substances may be included on the device surface by an appropriate method, such as, for example, adsorption, precipitation, ionic attachment, biotin/streptavidin interaction, covalent attachment, and/or any other appropriate attachment method.

In some exemplary embodiments, substances, such as aptamers, may be covalently attached, directly or indirectly, to the device surface. The surface may be composed of a metal alloy, silanes, polyurethane, cellulose or any other polymeric material. A device surface may include attachment sites, such as chemically reactive sites, which may be utilized to covalently link substances to the device surface. Chemically reactive sites, such as, for example, free hydroxyl, carbonyl, sulfhydryl, carboxyl or amine surface groups, may be used to chemically react with an appropriate reactive linker molecule to form a covalent attachment. Chemically reactive sites may be generated on a device surface by a variety of methods, which may include, but are not limited to oxidation, reductive amination, esterification, acetylation, acetalization, salt formation, coronal discharge, etching, enzymatic modification, photoreduction, and/or any other appropriate method. In some embodiments, the substance may be directly coupled to the surface. In other embodiments, a substance may be linked to the surface via another substance, such as a crosslinking agent, a priming agent or a coupling agent.

In an exemplary embodiment, an aptamer may be linked to the device surface by, for example, a cross-linking agent. Nucleic acids may be modified to include particular reactive functionalities, such as, for example, 3′ or 5′ amino groups. The reactive sites on the nucleic acids may then be reacted with an appropriate cross-linking agent, priming agent, or coupling agent, such as, for example, divinyl sulfone (DVS), silanes, succinmidyl esters, maleimides, imidoesters, halogenating agents, pyridyl disulfides, EDC reaction (e.g., 1-Ethyl-3-[3-dimethylaminopropyl]carbodiimide hydrochloride) photoreactive cross-linkers, and/or any other appropriate cross-linking agent, which may be attached to the device surface. Cross-linking agents may also be utilized to enhance chemical stability. They may also space the substance further from the device surface, reduce steric hindrances or repulsion from the device surface, and may also generate a greater freedom of movement of the substance such that it may localize and bind to a target in the body tissue more effectively.

In another embodiment, a library of aptamers approaching the number of possible binding sites found in the ECM may be coated onto at least a portion of a surface of an implant, the molar ratio of all the different aptamers may be approximately one. The molar ratio of the aptamers may also represent the molar equivalence of the binding sites found in ECM.

In another embodiment, the chemical stability of the aptamer coating on the implant surface may be enhanced by co-immobilizing with other polymeric molecules, such as synthetic or natural polymers at a molar ratio of at least 10 or less.

In another embodiment, examples of which are shown in FIGS. 3a, 3b, 3c, 3d and 3e, unique molecules may also include multimeric or chimeric aptamers, which may include multiple binding sites for at least one target. For example, a chimeric aptamer may be generated from two or more aptamers joined by a linking sequence which may include, for example, an oligonucleotide sequence or other polymeric linkage. In some embodiments, multimeric aptamers may be generated utilizing, for example, rolling circle amplification, such as from a circular DNA template, and/or any other appropriate method. A chimeric aptamer may, for example, be utilized to bind multiple targets in the body tissue. FIG. 3a illustrates a homo-bifunctional aptamer 50a with identical aptamers 12. FIG. 3b illustrates a heterobifunctional aptamer 50b with different aptamers 12, 12′. FIG. 3c illustrates a homo-n-functional aptamer 50c with identical aptamers 12. FIG. 3d illustrates a hetero-n-functional aptamer 50d with at least multiple aptamers 12, 12′. FIG. 3e illustrates an aptamer construct 50e including an aptamer 12 attached to another molecule 15, such as, for example, a protein, enzyme, and/or any other appropriate molecule. A chimeric aptamer may also be utilized to bind at one site to the body tissue and at another site to the device surface and/or other substance present on the device surface, such as a cross-linking molecule. A desirable coating may also include, for example, a chimera of an aptamer and a catalytically active ribozyme. Similarly, coatings including deoxyribozymes (DNAzymes) may also be desirable.

In another aspect, substances may be spatially arranged on the surface of the device to aid in attachment of the device to the body tissue. In some embodiments, devices may be present at tissue boundaries during implantation. The device may then be functionalized with substances, such as aptamers, wherein the differences in the tissue composition across the boundary may be utilized to determine the placement of particular substances on the device surface. For example, a device may be present at a boundary between two layers or regions of tissue with different ECM compositions. One side of the device may then be functionalized with one set of substances (e.g. aptamers) selected for binding to one layer or region of tissue and the other side of the device may be functionalized with a different set of aptamers or other molecules for binding to the other layer or region. The device may also be functionalized to bind to different layers or regions of tissue utilizing the same ECM molecular composition that varies in relative abundance of ECM components across a boundary by, for example, varying the relative abundance of aptamers on the surface of the device. This may, for example, include utilizing a higher abundance of an aptamer on one portion of the device and a lower abundance on another portion such that the higher abundance portion may bind to a corresponding tissue region or layer with a higher abundance of a target ECM component.

In some embodiments, a particular substance may also be substantially evenly distributed on at least a portion of the surface of the device to increase the probability of binding to a target in the body tissue.

### EXAMPLE 1

A predominant ECM protein, fibronectin, was chosen as a model target for development of novel DNA aptamers. A SELEX protocol, in which all that is required is a method for facile partitioning of the bound nucleic acids from the free or weakly binding species, was utilized to generate aptamers. Between multiple rounds of such partitioning, polymerase chain reaction (PCR) amplification of the binding fraction was performed to produce the desired enrichment. A target protein was passively adsorbed to 0.3 μm polystyrene beads. To develop fibronectin aptamers, the initial aptamer pool consisted of approximately 10¹⁵ randomized nucleic acid sequences of 35 nucleotides flanked by constant regions for PCR priming (e.g. 5′-ForwardPrimer-N35-ReversePrimerNot-3′). Asymmetric PCR was then performed using a 100-fold excess of forward to reverse primer. This results in a largely single-stranded DNA pool for the next round of binding. Prior to multiple rounds of SELEX, the initial library was exposed to “naked” polystyrene beads to clear it of non-specific binders. In some aptamer development, the beads were optionally “blocked” with nonfat dry milk after fibronectin adsorption to cover any vacant polystyrene surface. All binding was performed in 20 mM Tris buffer, 100 mM NaCl, at pH 7.4. For brevity, the major steps in a single round of selecting fibronectin aptamers were: 1) A library of ssDNA ligands was exposed to polystyrene with immobilized fibronectin, 2) Binding occurred for 30 min at 37° C., 3) Wash with buffer to remove “weak” binders, 4) “Strong” binders were removed by elution with 7M urea, 5) Recovered nucleic acids were re-amplified by asymmetric PCR, 6) Proceed to next round of binding. The volume of the wash increased with each round of selection. An increase of binding to fibronectin of the enriched ligand pool was seen in comparison with the randomized starting library.

To determine if the nucleic acid pool for ligands to fibronectin was being enriched through the developed SELEX protocol, simple batch-binding assays were performed, and supernatants were analyzed for nucleic acids using UV-vis spectrophotometry by a 1 μl Nanodrop™ spectrophotometer. While only 5% of the randomized starting library remained bound when presented to the fibronectin immobilized on beads, after 6 rounds of enrichment, 75-82% of the selected pool did bind to fibronectin. To obtain a more quantitative characterization of affinity, fluorescence polarization experiments in free solution were also performed. Fluorescence anisotropy measurements provide information on molecular orientation and mobility and processes that modulate them, such as receptor-ligand interactions, and protein-DNA interactions. Upon excitation with plane polarized light, fluorophore molecules with their absorption transition vectors aligned parallel to the electric vector of the polarized light are selectively excited. When the fluorophores are attached to small, rapidly rotating molecules (such as an aptamer), the initially photoselected orientational distribution becomes randomized prior to emission, resulting in low fluorescence anisotropy. Conversely, binding of the low molecular weight labeled molecule to a large, slowly rotating molecule (such as a protein) results in high fluorescence anisotropy.

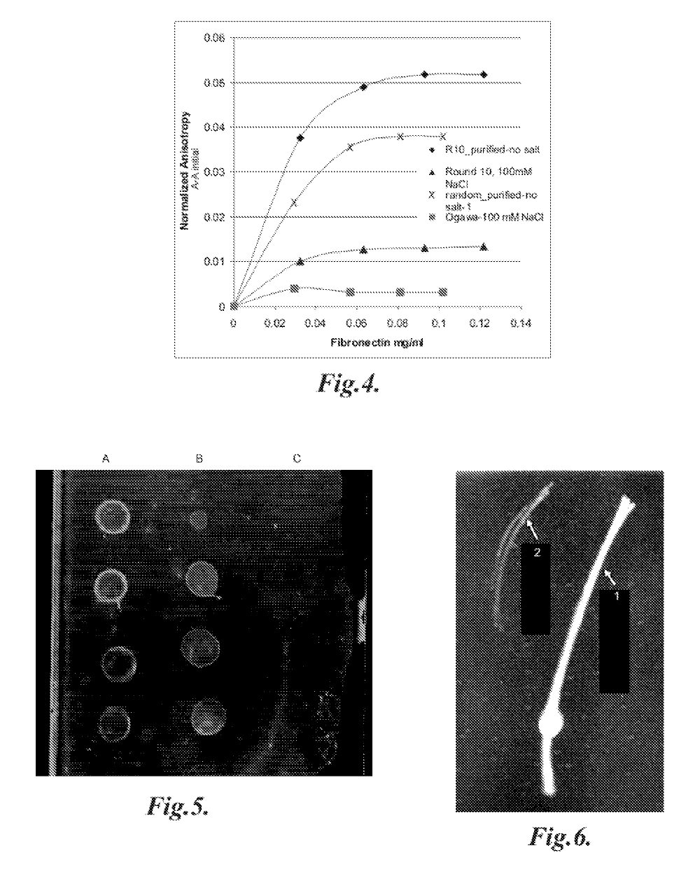

Fluorescence anisotropy therefore provides a direct readout of the extent of binding and complex formation. As shown in FIG. 4, after 10 rounds of selection, a pool of aptamer ligands with considerably enhanced affinity for fibronectin over the initial random pool was generated.

Using a “dot-blot” type format with fibronectin, bovine serum albumin (BSA), and nonfat dry milk proteins immobilized on a nitrocellulose coated slide (FAST slide, Whatman), binding and specificity was assessed in a standard microarray scanner. After spotting dilution series of the 3 proteins, the slide was also “blocked” with BSA. The fibronectin aptamers developed after 10 rounds of SELEX have more affinity for fibronectin, row A, than for BSA, row B, and no detectable affinity for a large number of milk proteins, row C, as shown in FIG. 5. Without wishing to be bound by theory, the cross-reactivity with the BSA spots in row B (and background binding to the remainder of the blocked slide) may be explained by the relatively broad specificity of the aptamer to fibronectin due to its large epitope. Fibronectin and BSA also have very similar net charge (isoelectric point or pI of ˜5.0 and 4.7 respectively. Negative selection may be employed to clear the pool of BSA-binding aptamers.

### EXAMPLE 2

In order to demonstrate immobilizing aptamers on a device, a cellulose membrane was modified with an aptamer that was functionalized with 5′-amino groups and conjugated to the membrane (thickness 20 microns, diameter 210 microns) made of regenerated cellulose through divinyl sulfone conjugation chemistry. After immobilization, the presence of the aptamer on the sensor membrane surface was verified by reaction with a DNA specific dye (SYBR gold, Invitrogen), as shown in FIG. 6. The difference in fluorescence between an aptamer-coated membrane 1 and a control unmodified membrane 2 also bathed in SYBR gold is clearly evident as shown.

The embodiments and examples described above are intended to be illustrative and not limiting. It will be appreciated by those of ordinary skill in the art that other embodiments of the present invention are possible without departing from the spirit or essential character of the invention hereof. The scope of the present invention is indicated by the appended claims, and all changes that come within the meaning and range of equivalents thereof are intended to be embraced therein.

1. A method for increasing the biocompatibility between an implantable device and a biological matter, comprising:
functionalizing said device by attaching at least one unique molecule to at least a portion of at least one surface of the device;
wherein said at least one unique molecule specifically binds to a binding site of at least one tissue component of said biological matter.

2. The method of claim 1, further comprising attaching a diverse library of aptamers to said at least a portion of said at least one surface, wherein the diversity of said library approaches the diversity of complementary binding targets in said at least one tissue component.

3. The method of claim 1, wherein said unique molecule comprises a DNA aptamer, a RNA aptamer or a combination thereof.

4. The method of claim 3, wherein said at least one aptamer is selected using a systematic evolution of ligands by exponential enrichment (SELEX) protocol.

5. The method of claim 1, wherein said unique molecules are capable of attachment to said device surface by adsorption, precipitation, ionic attachment, covalent attachment, or combinations thereof.

6. The method of claim 5, wherein said covalent attachment comprises using at least one cross-linking agent.

7. The method of claim 6, wherein said at least one cross-linking agent comprises divinyl sulfone (DVS), silanes, succinmidyl esters, maleimides, imidoesters, halogenating agents, pyridyl disulfides, EDC reaction (e.g. 1-Ethyl-3-[3-dimethylaminopropyl]carbodiimide hydrochloride), photoreactive cross-linkers, or combinations thereof.

8. The method of claim 2 wherein said aptamers are present in proportions relative to the relative abundances of the complementary binding targets in the body tissue.

9. The method of claim 3 wherein said aptamer binds to known or unknown cell receptors in a biomimetic manner.

10. The method of claim 1 further comprising complementing said unique molecules with polymeric molecules to sterically block degradation of the unique molecules.

11. The method of claim 10 wherein said polymeric molecules comprise polyethylene glycols, polyurethanes, silanes, polysaccharides, biopolymers or combinations thereof.

12. The method of claim 1 wherein said at least one unique molecule comprises homofunctional aptamers, heterofunctional aptamers, n-functional homo aptamers, n-functional hetero aptamers, aptamer-ribozyme constructs, aptamer-DNAzyme constructs, aptamer-peptide constructs, or combinations thereof.

13. The method of claim 1 further comprising functionalizing by attaching at least a second unique molecule to at least a portion of a second surface of the device, wherein said first unique molecule and second unique molecule are capable of binding to different body tissues or regions of tissue.

14. A method for increasing the biocompatibility of a device, comprising:
attaching at least one unique molecule to at least a portion of a surface of the device in a spatial distribution for increasing the probability of binding to at least one target in a body tissue, wherein said unique molecule comprises an aptamer.

15. The method of claim 14, wherein said spatial distribution is determined by predicted or measured locational abundance of said target in the body tissue.

16. The method of claim 14 wherein said device is functionalized to be present at a boundary between two body tissues or regions of tissue during implantation.

17. The method of claim 16 wherein the placement of said aptamers on said at least one portion of the surface of the device is determined by the differences in the tissue composition across the boundary.

18. The method of claim 16 wherein said functionalizing comprises attaching two different aptamers to the surface of the device, said first aptamer and said second aptamer are capable of binding to different body tissues or regions of tissue.

19. The method of claim 16 wherein said functionalizing comprises attaching an aptamer to said at least one portion of the surface of the device, said aptamer is capable of binding to two different body tissues.

20. An implantable device with improved biocompatibility with biological matter, comprising:
at least one portion of at least one surface functionalized with at least one unique molecule for binding to various components of a biological tissue, wherein said unique molecule comprises an oligonucleotide.

21. The device of claim 20 wherein said at least one unique molecule is attached to said at least one surface with non-nucleotide molecules.

22. The device of claim 20 wherein said oligonucleotides are capable of binding to two different biological tissues or regions of tissue.

23. The device of claim 20 wherein said oligonucleotides comprise DNA aptamers, RNA aptamers or combination thereof.

24. The device of claim 20 further comprising a diverse library of oligonucleotides on said at least a portion of said at least one surface, wherein the diversity of said library approaches the diversity of complementary binding targets in said biological tissue.
